(19) United States
(12) Patent Application Publication
(10) Pub. No.: US 2003/0021782 A1
(43) Pub. Date: Jan. 30, 2003

(54) Chlamydia infection

(76) Inventors: Cho-Chou Kuo, Seattle, WA (US); Tsun-Mei Lin, Tainan (TW); Lee Ann Campbell, Seattle, WA (US); Michael E. Rosenfeld, Seattle, WA (US)

Correspondence Address: ROYLANCE, ABRAMS, BERDO & GOODMAN, L.L.P., 1300 19TH STREET, N.W., SUITE 600, WASHINGTON,, DC, 20036, US

(21) Appl. No.: 09/910,920

(22) Filed: Jul. 24, 2001

(51) Int. Cl. A61K 39/395

(52) U.S. Cl. 424/145.1

(57) ABSTRACT

Chlamydia binds to the mannose-6-phosphate receptor on mammalian cells. Molecules that prevent binding of elementary bodies to the mammalian cells can be therapeutically beneficial in treating and/or preventing Chlamydial infection.

Portions of the research described herein were supported in part by monies from the National Institutes of Health.

### BACKGROUND OF THE INVENTION

Chlamydia are obligate intracellular bacteria. The extracellular form consists of infectious elementary bodies (EB) that gain entry into target cells, convert into reticulate bodies and proliferate therein.

It is believed that there is a recognition means at the surface of target cells that enables the EB to recognize and to bind a target cell. The cell surface receptor for the EB is one that likely interacts with the major outer membrane protein (MOMP) of the EB. The MOMP is the principal structural protein of the EB. The MOMP is glycosylated. The carbohydrate structure of the MOMP glycom is a high mannose type ogliosaccaride (Kuo et al. J. Clin. Invest. 98: 2813-8,1996).

Once the EB enters into the cell by mechanisms unknown, the EB is able to survive within the host cell without degradation. Chlamydia are able to develop and to multiply in the cytoplasm of mammalian host cells within membranous vesicles called inclusions.

### SUMMARY OF THE INVENTION

An object of the instant invention is to identify the cell surface receptor which enables Chlamydia to bind to a mammalian target cell. Molecules that bind to the receptor and occupy the receptor can be used to intercede and to prevent adsorption of the EB to the surface of target cells.

Another object of the instant invention relates to the identification of the mechanism by which the EB enters the cell. Identification of that mechanism enables use of various molecules to control expression of that cellular mechanism and that would render the Chlamydia susceptible to intracellular degradation.

Yet another object of the invention is to identify those structures on the EB that enable binding to the target cell. Molecules that bind to the EB can be used to prevent binding of the EB to a target cell.

Another object of the invention is to identify other molecules and mechanisms that modulate Chlamydia binding to a target cell, entry into a cell and propagation in a cell. Disruption or control of the other molecules and mechanism can minimize Chlamydia proliferation. For example, cytokine, such as insulin-like growth factor-2, and such molecules.

Those and other objects of the instant invention have been attained by the discovery of a receptor for Chlamydia at the surface of mammalian target cells and of the intracellular structure by which the EB enters into and survives within the cell. Moreover, that receptor binds another molecule that impacts the process.

Thus, the instant invention includes materials and methods for inhibiting the binding and entry of Chlamydia into mammalian target cells, as well as materials and methods for preventing the proliferation of Chlamydia in a mammalian target cell.

### DETAILED DESCRIPTION OF THE INVENTION

Three commonly known species of Chlamydia include C. trachomatis, C. psittaci and C. pneumoniae.

C. trachomatis is a problematic pathogen known for causing trachoma, a prevalent ocular disorder. C. trachomatis also is associated with venereal disease. C. trachomatis can be found in the urinary tract as well as in the cervix, uterus and fallopian tube.

C. psittaci causes a pneumonia-like disorder.

C. pneumoniae causes respiratory infection and is associated with cardiovascular disease. The association with cardiovascular disease was first demonstrated by detection of organisms in atheromatous lesions. The cells in atherosclerotic lesions containing C. pneumoniae include macrophages, smooth muscle cells and endothelial cells.

C. trachomatis infects macrophages. Macrophages express a mannose receptor.

Monocytes, such as macrophages, can be resident throughout the body. Monocytes produce a number of different cytokines that have local and distal effects on various cells. Some of the cytokines produced by monocytes include interleukins, interferons, insulin-like growth factor-1, insulin-like growth factor-2, chemotactic proteins and other growth factors.

For example, insulin-like growth factor-2 (IGF-2) is produced by human monocytes and enhances the susceptibility of human endothelial cells to infection by C. pneumoniae. Thus, monocytes or macrophages present in the arterial wall may, through paracrine interactions of secretory products released thereby, stimulate infection of endothelial cells by Chlamydia.

Carbohydrates appear to have a key role in the bacteria-cell recognition process. Thus, the cell surface receptor and the EB may have complementary structures that allow for recognition and binding.

The mannose-6-phosphate (Man-6-P) receptor and mannose receptor are cell surface molecules. The Man-6-P receptor is multifunctional and is known to target lysosomal enzymes. The receptor is known to have a binding site for phosphomannosyl residues as well as for IGF-2. The two ligands can bind the receptor simultaneously and non-competitively.

The Man-6-P receptor is found on the Golgi apparatus and functions in transporting lysosomal enzymes to the lysosomes. Lysosomal enzymes are rich in mannose and thus, those intracellular enzymes also are ligands of the Man-6-P receptor.

The receptor also is found on the surface of plasma membranes in clarthrin-coated pits and likely functions to internalize IGF-2 and extracellular lysosomal enzymes. When internalized, IGF-2 is degraded in the endosomomal compartment where internalized lysosomal enzymes are delivered to the lysosomes.

The EB has surface structures that contain mannose residues. Whether mannose or mannose-6-phosphate, the EB binds to endothelial cells presumably via the mannose-6-phosphate receptor; the mannose receptor; or a receptor that interacts with or is impacted by activity of the mannose-6-phosphate receptor or mannose receptor. It can be seen, then, that the binding of Chlamydia organisms, and binding of IGF-2 to the Man-6-P receptor at the cell surface facilitates entry of the EB into the cell. Occupancy of the Man-6-P receptor inhibits the uptake of lysosomal enzymes into endocytic vesicles. That enables the endocytosed Chlamydia to survive in the cell and provides Chlamydia the opportunity to multiply within phagosomes.

Phagosomes manipulated to express additional vacant Man-6-P receptors would enable binding of lysosomal enzymes to the phagosome and hence, destruction of the contents thereof, including the reticulate bodies of the Chlamydia organisms. Over-expression of Man-6-P receptors can occur through manipulation of the promoter thereof or replacement thereof by other stronger promoters. That can be achieved, for example, by addition of additional Man-6-P receptor genes or by homologous recombination, techniques know in the art.

Alternatively, the gene encoding the Man-6-P receptor can be cloned and expressed to yield isolated receptor which can be modified without destroying the ability of the molecule to bind to the EB surface. The Man-6-P receptor molecules can be used as adsorbents that bind to the EB. Nielsen, Prog. Growth Fact. Res. 4:257-290, 1992; Nissley & Lopaczynski, Growth Factors 5:29-43,1991.

Molecules that engage the Man-6-P receptor can be used to prevent binding of the EB to the cell surface. The molecules used to intervene in the binding of Chlamydia to the host cell can be of any origin, natural or synthetic, so long as the molecule engages the receptor and ultimately prevents binding of Chlamydia to the cell surface. Hence, at the simplest level, monomeric or polymeric mannose-6-phosphate can be used to intercede in the binding reaction. The mannose-6-phosphate, whether monomeric or polymeric, can be modified to enhance in vivo properties without minimizing the biological activity of binding to the receptor and preventing binding of Chlamydia to the cell surface. For example, one approach is to substitute an oxygen for a carbon atom in the hexose ring yielding a metabolically more stable molecule that resides in the body for a longer period of time before being metabolized.

Alternatively, other mimetics which simulate the steric configuration of mannose-6-phosphate and that bind to the Man-6-P receptor can be produced from molecules other than mannose-6-phosphate or, for that matter, other than carbohydrates, to serve as molecules that bind to the Man-6-P receptor. Such mimetics include carbohydrates other than mannose, amino acids, nucleic acids, other organic molecules and the like. An artisan can determine the basic spatial and steric configuration of molecules that bind to the Man-6-P receptor and synthesize molecules having that configuration for use as a Man-6-P mimetic using known materials and methods.

Another means to minimize binding of the EB to the cell surface is to generate a molecule that binds to the EB surface thereby hindering the ligand present on the EB surface from engaging the Man-6-P receptor. For example, the configuration of the Man-6-P receptor that provides the structure to which the Man-6-P ligand of the EB engages can be determined. Then, using rational design programs, a molecule that simulates the Man-6-P ligand pocket can be configured and synthesized to serve as a means to bind to the surface of the EB and thereby prevent binding of the EB to the cell surface of the target cell.

Another approach would be to use antibodies to the EB or to the Man-6-P receptor that blocks binding to and entry of EB into the target cell. Antibodies, monoclonal or polyclonal, and antigen binding proteins thereof, whether derived from natural molecules or constructed recombinantly, can be made using techniques known in the art.

For example, antibody that binds specifically to mannose-6-phosphate can be made. Either a polymeric Man-6-P or other immunization schemes can be used, for example, using a carrier and/or adjuvant. Such an antibody would bind to the EB to prevent engaging same to the receptor.

Alternatively, antibody can bind to the Man-6-P receptor. Either whole cells expressing receptor, membranes expressing receptor or isolated receptor can be used as immunogen. Such antibody would bind to the target mammalian cell. Because the receptor has two binding sites for Man-6-P and to IGF-2, an antibody of a polyclonal preparation or a monoclonal antibody can be directed solely to one or to the other binding site. Thus, an antibody can bind only to the Man-6-P binding site of the Man-6-P receptor or only to the IGF-2 binding site of the Man-6-P receptor.

Because IGF-2 impacts Chlamydia infectivity and proliferation, manipulating IGF-2 metabolism can be beneficial. Thus, by controlling binding of IGF-2 to the Man-6-P receptor, Chlamydia infectivity can be impacted. For example, antibody binding to IGF-2 can be used to avoid IGF-2 binding to the Man-6-P receptor. Another antibody would be one that binds to the IGF-2 binding site of the Man-6-P receptor as described above.

In cases of over expression of IGF-2 or the Man-6-P receptor that result in enhanced EB binding to the cell, and susceptibility to infection, means to down regulate the expression of IGF-2 or of the Man-6-P receptor can be used.

Hence, the instant invention provides pharmaceutic compositions for treating disorders arising from Chlamydial infection, such as optic disorders, sexually transmitted diseases, respiratory diseases and the like, comprising an amount of a molecule sufficient to bind to the mannose-6-phosphate receptor on the surface of mammalian cells or to the mannose-6-phosphate ligand present on the Chlamydial EB and a biologically acceptable carrier, diluent or excipient. The composition can be used both in vitro and in vivo.

The instant invention also provides for compositions comprising molecules that bind to IGF-2 or to the IGF-2 receptor and a biologically acceptable carrier, excipient or diluent.

The instant invention also provides for compositions comprising molecules that alter expression of the Man-6-P receptor, such as a molecule that regulates expression of the receptor or an anti-sense molecule that prevents expression of the receptor, and a biologically acceptable carrier, excipient or diluent.

The composition comprises an effective amount of the active ingredient. That particular amount can be determined without resort to undue experimentation by practicing normal pharmaceutic methods of establishing dose-response relationships in suitable animal models and then extrapolating to humans. The normal procedures for determining proper dosage as mandated by government regulations, such as, safety and efficacy studies, are practiced without resort to undue experimentation using methods known in the art.

As is known in the art, dosages are modified based on the particular medical application, such as the severity of the disease, the weight of the subject, age of the subject, half-life in circulation, route of administration and so on. That information, such as dosage, number of doses, route of administration and so on may vary from subject to subject and can be determined readily by one of ordinary skill in the art.

The active ingredient can be administered in a variety of ways depending on the malady. For example, in a case of a trachoma, an artisan might consider not only a parenteral formulation but also non-parenteral means including eye drops. In the case of a sexually transmitted disease, a local formulation including an ointment or cream may be considered. In the case of a respiratory disorder, a non-parenteral means includes a nebulizer and the like.

In each case, the formulation can be configured using appropriate specific amounts of active agent and appropriate diluents, excipients or carriers.

The invention now will be exemplified by way of the following non-limiting example.

C. pneumoniae strain AR-39, a respiratory isolate (Kuo et al., J. Clin. Micro. 24:1034-7, 1986) was propagated in HL cells (Kuo & Grayson, J. Inf. Dis. 162:755-8, 1990). Harvested organisms were purified by Hypaque gradient centrifugation. Purified organisms were resuspended in Chlamydial transport medium, sucrose-phosphate-glutamic acid, and stored at −75° C. in small aliquots until use.

HMEC-1 is a transformed human arterial endothelial cell line (Ades et al., J. Invest. Derm. 99:683-90, 1992). Human macrophage U937 (ATCC CRL 1593) cells are non-adherent to the surface of glass or plastic culture vessels. Generally, the media used for culturing those cells were endothelial cell basal medium (Clonetics, San Diego, Calif.) supplemented with 2% fetal calf serum for HMEC-1 cells, and RPMI-1640 (RPMI, GIBCO Laboratories, Grand Island, N.Y.) supplemented with 10% fetal calf serum for U937 cells.

To prepare conditioned medium, U937 cells were maintained in RPMI medium supplemented with 10% fetal calf serum for several passages. Cells at a density of 1×10⁶ cells/mL were cultured with RPMI containing 2% fetal calf serum or serum free macrophage culture medium (GIBCO Laboratories, Grand Island, N.Y.) for 1 to 3 days. Culture supernatant (conditioned medium) was collected and cleared of cells by centrifugation at 700×g for 10 mm and stored at −20° C. for subsequent assays.

Data from a series of initial experiments shows that: 1) monocytes cultured in serum-free macrophage culture medium produce higher titers of infectivity enhancing factors than monocytes cultured with RPMI supplemented with 2% fetal calf serum; 2) the amount of infectivity enhancing activity secreted is correlated inversely with the length of time in culture; and 3) the seeding density of U937 cells for secretion of the maximum amount of enhancing activity is 1×10⁶ cells/mL. Therefore, conditioned medium harvested from 24 hour cultures of 1×10⁶ cells/mL in serum-free medium were used for all subsequent experiments.

To assay for growth enhancing activity, conditioned medium, fractions of conditioned medium, insulin-like growth factor-1 (IGF-1) and IGF-2 were tested in HMEC-1 cell culture for enhanced susceptibility to C. pneumoniae. HMEC-1 cells were trypsinized from stock cultures and plated to achieve a cell monolayer in 24 h in 24-well culture plates. A 12 mm diameter glass coverslip was placed in each well for growing cells for ease of staining for microscopic observation and counting inclusions for assaying titers. For inoculation of cell culture, the culture fluid was removed and the cell monolayer washed three times with Hank's balanced salt solution. Two-tenth mL of inoculum were added per well and the plate was centrifuged for 1 h at 700 to 800× g at 25° C. After centrifugation, the inocula were removed, 1 mL of specified culture medium was added, and the plates were incubated in a CO₂ incubator at 35° C. for 3 days. After incubation, coverslips were removed, fixed with methanol and stained with a Chlamydia genus-specific monoclonal antibody conjugated with fluorescein isothiocyanate. Inclusions were counted in 30 random fields at ×400 magnification. The infectivity titers were expressed as inclusion forming units (ifus) per milliliter, which had been adjusted for dilution factor and inoculum size (Furness et al., J. Gen Micro. 23:213-9, 1960). Triplicate coverslips were counted in each experiment. Experiments were repeated 2 or 3 times.

Characterization of Chlamydial growth enhancing factors was carried out as follows. Conditioned medium of a one day old culture of U937 cells in RPMI supplemented with 2% fetal calf serum was used. Conditioned medium was heated at 70° C. for 2 h and tested for growth enhancing activity against AR-39 in HMEC-1 cells. An equal volume of conditioned medium was mixed with fresh endothelial basal medium containing 2% fetal calf serum for culturing infected HMEC-1 cells.

The enhancing activity was completely abolished by heating the conditioned medium at 70° C. for 2 h, suggesting that the enhancing factors are likely proteinaceous in nature.

For determining molecular size, one day old conditioned medium of U937 cells cultured in serum-free macrophage culture medium was fractionated into molecular weight ranges of larger than 30,000, 30,000 to 10,000, 10,000 to 5,000 and smaller than 5,000 using three different pore size filters, Biomax-30, Biomax-10 and Biomax-5 ultrafilters (Millipore, Bedford, Mass.) with the molecular weight cut-off of 30,000; 10,000; and 5,000, respectively. For size fractionation, a starting volume of 200 mL of conditioned medium was used. Each fraction was concentrated to 2 mL (a 100× concentration). The remaining filtrate which had been passed through the 5,000 molecular weight pore size filter also was saved for testing. That fraction and the starting conditioned medium were tested without concentration. Fractions were sterilized by filtration through a 0.22 μm Millipore filter and stored at −75° C. until assayed for growth enhancing activity. In this assay, inoculated cells were cultured with endothelial cell basal medium supplemented with equal volume of fractionated conditioned medium. Control cultures were incubated with endothelial cell basal medium.

The infectivity enhancing factor was identified in the fraction containing molecular masses between 5,000 to 10,000.

To test the growth enhancing activity of IGF-1 and IGF-2, inoculated cells were cultured with endothelial cell basal medium alone or basal medium supplemented with IGF-1 or IGF-2 at serial concentrations. Recombinant human IGF-1 and IGF-2 were purchased from R & D Systems, Inc. (Minneapolis, Minn.).

Human recombinant IGF-1 or IGF-2 were added to the culture medium at concentrations of 1, 5 and 10 ng/mL. IGF-2, but not IGF-1, enhanced the growth of C. pneumoniae strain AR-39 in HMEC-1 cells. A dose response to IGF-2 was conducted at IGF-2 concentrations of 0.1, 0.5, 1.0, 2.5, 5 and 10 ng/mL concentrations. The enhancing activity was noted as low as 0.5 ng/mL (35% increase, p<0.05, N=3), and increased at 5 ng/mL (256% increase, p<0.001). Concentrations of IGF-1 and IGF-2 tested were not grossly toxic to HMEC-1 cells.

Antibody neutralization of IGF activity in conditioned medium was tested using goat anti-human IGF-1 and IGF-2 antibodies (R & D Systems, Inc) in a blocking assay. The conditioned medium fraction containing the enhancing activity was incubated with anti-human IGF-1 or anti-human IGF-2, at a single concentration of 10 μg/mL for anti-IGF-1 and 1 μg/mL for anti-1 GF-2 antibody for 1 h at 37° C. in 24-well culture plate. Anti-IGF antibodies were used at 2 times the 50% neutralization dose (ND₅₀) for each antibody as suggested by the manufacturer. Following preincubation, the neutralized medium was mixed with equal volume of fresh endothelial cell basal medium for incubation of HMEC-1 cells infected with AR-39. Infectivity titers were assayed after 3 days of incubation as described above.

To confirm that IGF-2 accounted for most of the infectivity enhancing activity, antibodies specific to human IGF-1 and IGF-2 were used to neutralize the enhancing activity in the U937 cell conditioned medium. Anti-IGF-2, but not anti-IGF-1, neutralized a majority of the enhancing activity.

All references cited herein are herein incorporated by reference in entirety.

It will be appreciated that various modifications can be made to the teachings set forth herein without departing from the spirit and scope of the instant invention.

1. A composition comprising:
(a) a Chlamydia infection inhibiting amount of a molecule that interacts with mannose-6-phosphate, mannose-6-phosphate receptor or insulin-like growth factor-2; and
(b) a pharmaceutically acceptable carrier, diluent or excipient.

2. The composition of claim 1, wherein said molecule is an antibody.

3. The composition of claim 2, wherein said antibody specifically binds to mannose-6-phosphate.

4. The composition of claim 2, wherein said antibody specifically binds to a mannose-6-phosphate receptor.

5. The composition of claim 2, wherein said antibody specifically binds to insulin-like growth factor-2.

6. The composition of claim 4, wherein said antibody binds to the mannose-6-phosphate binding site.

7. The composition of claim 4, wherein said antibody binds to the insulin-like growth factor-2 binding site.

8. The composition of claim 1, wherein said molecule comprises mannose-6-phosphate.

9. A method of reducing infectivity of Chlamydia comprising administering to a host exposed to Chlamydia, a composition comprising a molecule that interacts with mannose-6-phosphate, mannose-6-phosphate receptor or insulin-like growth factor-2 to reduce Chlamydia infectivity.

10. The method of claim 9, wherein said molecule is an antibody.

11. The method of claim 9, wherein said antibody specifically binds to mannose-6-phosphate.

12. The method of claim 9, wherein said antibody specifically binds to a mannose-6-phosphate receptor.

13. The method of claim 9, wherein said antibody specifically binds to insulin-like growth factor-2.

14. The method of claim 12, wherein said antibody binds to the mannose-6-phosphate binding site.

15. The method of claim 12, wherein said antibody binds to the insulin-like growth factor-2 binding site.

16. The method of claim 9, wherein said molecule comprises mannose-6-phosphate.
